(19) United States
(12) Patent Application Publication
(10) Pub. No.: US 2020/0246322 A1
(43) Pub. Date: Aug. 6, 2020

(54) METHOD FOR TREATING GLUTEN-RELATED ALLERGIES AND INTOLERANCE ISSUES WITH LOPERAMIDE

(71) Applicant: Arpad Sooky, Aliquippa, PA (US)

(72) Inventor: Arpad Sooky, Aliquippa, PA (US)

(21) Appl. No.: 16/266,714

(22) Filed: Feb. 4, 2019

(51) Int. Cl. A61K 31/451; A61P 37/08; A61P 1/12

(52) U.S. Cl. CPC A61K 31/451; A61K 9/0053; A61P 1/12; A61P 37/08

(57) ABSTRACT

A method for treating gluten-related allergies and gluten intolerance is provided. The method includes administering to a patient an effective amount of a drug having loperamide as an active ingredient; specifically, wherein the patient suffers from gluten-related allergies and/or gluten intolerance.

### BACKGROUND OF THE INVENTION

The present invention relates to methods of treating gluten allergies and intolerance and, more particularly, to a new use of loperamide as an agent for treating gluten-related allergies and gluten intolerance.

The gluten-related allergies and gluten intolerance is approaching endemic proportions, and is usually life-changing if not debilitating. There are no other methods to treat gluten allergy/intolerance, besides dietary restriction/elimination of gluten. Dietary restrictions/changes require lifestyle changes, and leave the gluten intolerant subject to inadvertent accidental exposure to gluten, which can result in anything from discomfort to severe suffering and anaphylactic shock.

As can be seen, there is a need for a new use of loperamide as an agent for treating gluten-related allergies and gluten intolerance, which would be a boon since there currently are no effective medications for treating gluten-related intolerance and allergies.

Loperamide is an over the counter (OTC) medication used to decrease the frequency of diarrhea. The medication is taken by mouth. As a result, Loperamide is a non-patent protected, low-cost medicament available to everyone that the inventor has discovered a secondary medical application for treating gluten-related allergies and gluten intolerance.

### SUMMARY OF THE INVENTION

In one aspect of the present invention, a method of treating gluten-related allergies and/or gluten intolerance includes administering to a patient an effective amount of a drug comprising loperamide as an active ingredient.

In another aspect of the present invention, such administration is taken orally wherein titration is involved in finalizing the effective dosage, wherein the effective dosage starts with a standard lowest dosage of loperamide recommended to treat diarrhea comprising a 2 mg after a first loose stool, another 2 mg after each subsequent loose stool; and further comprising adding an additional 2 mg tablet every 6 hours, wherein the effective dosage is slowly increased so as to create a resistance to constipatory effects, wherein the effective dosage starts is 150 percent of a standard lowest dosage for treating diarrhea, wherein the effective dosage is 2 mg, or wherein the effective dosage is less then 2 mg.

These and other features, aspects and advantages of the present invention will become better understood with reference to the following description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method for treating gluten-related allergies and gluten intolerance, the method includes administering to a patient an effective amount of a drug having loperamide as an active ingredient; specifically, wherein the patient suffers from gluten-related allergies and/or gluten intolerance.

The present invention may include a new use of the existing OTC medication (generic: loperamide, brand: Immodium™) to be taken orally by patient suffering from gluten allergy/intolerance symptoms. The Loperamide is taken until the gluten allergy/intolerance symptoms are relieved.

The amount of loperamide needed to treat/prevent the symptoms of gluten intolerance can be quite a bit higher than the levels that can cause constipation. In that case, a slow increase in dosage may be necessary in order to create a resistance to the constipatory effects. Titration may be necessary to reach the proper dosage to eliminate the symptoms, starting preferably with the standard lowest dosage of loperamide recommended to treat diarrhea (2 mg tablet after the first loose stool, 1 tablet after each subsequent loose stool) and adding 1×2 mg tablet every 6 hours until stool consistency normalizes, at which point the symptoms of gluten intolerance will disappear completely, if it is the type of gluten intolerance that is treatable with loperamide. In the experience of the inventor, the rate of successfully treating/preventing gluten intolerance completely is 100%, as long as it is the type of gluten intolerance that responds to loperamide, and the proper daily dosage is adhered to once it is determined to be effective for the patient.

The instructions for loperamide dosage pertaining to the treatment of diarrhea specify that no more than 4×2 mg should be taken every 24 hours, but that may not be enough for gluten intolerance, especially if the patient has a history of taking medications that may have caused an increase in resistance to the family of medications. There are minimal side-effects of loperamide at higher doses. The only one that is the most concerning is potentially causing cardiac arrhythmia, but the studies and evidence that support this claim are not enough to be convincing, so more research is needed to verify it.

The dosage could encompass any opiate, opiate derivative, or loperamide derivative. furthermore, the above-mentioned treatment may be effective for treating leaky bowel syndrome, since gluten intolerance and leaky bowel syndrome can have similar symptoms and sometimes be misdiagnosed.

It should be understood, of course, that the foregoing relates to exemplary embodiments of the invention and that modifications may be made without departing from the spirit and scope of the invention as set forth in the following claims.

What is claimed is:

1. A method of treating gluten-related allergies and/or gluten intolerance includes administering to a patient an effective dosage of a drug comprising loperamide as an active ingredient.

2. The method of claim 1, wherein the effective dosage is administered orally.

3. The method of claim 1, wherein titration is involved in finalizing the effective dosage.

4. The method of claim 1, wherein the effective dosage starts with a standard lowest dosage of loperamide recommended to treat diarrhea comprising a 2 mg after a first loose stool, another 2 mg after each subsequent loose stool;
and further comprising adding an additional 2 mg tablet every 6 hours.

5. The method of claim 4, wherein the effective dosage is slowly increased so as to create a resistance to constipatory effects.

6. The method of claim 1, wherein the effective dosage starts is 150 percent of a standard lowest dosage for treating diarrhea.

7. The method of claim 1, wherein the effective dosage is 2 mg.

8. The method of claim 1, wherein the effective dosage is less then 2 mg.

9. A method of treating leaky bowel syndrome includes administering to a patient an effective dosage of a drug comprising loperamide as an active ingredient.

10. The method of claim 9, wherein the effective dosage is administered orally.

11. The method of claim 9, wherein titration is involved in finalizing the effective dosage.

12. The method of claim 9, wherein the effective dosage starts with a standard lowest dosage of loperamide recommended to treat diarrhea comprising a 2 mg after a first loose stool, another 2 mg after each subsequent loose stool; and further comprising adding an additional 2 mg tablet every 6 hours.

13. The method of claim 12, wherein the effective dosage is slowly increased so as to create a resistance to constipatory effects.

14. The method of claim 9, wherein the effective dosage starts is 150 percent of a standard lowest dosage for treating diarrhea.

15. The method of claim 9, wherein the effective dosage is 2 mg.

16. The method of claim 9, wherein the effective dosage is less then 2 mg.
